(19) United States
(12) Patent Application Publication
(10) Pub. No.: US 2026/0315046 A1
(43) Pub. Date: Oct. 15, 2026

(54) BIOPSY PROBE INSERTABLE INTO A BODY

(71) Applicant: VPIX MEDICAL INCORPORATION, Daejeon (KR)

(72) Inventors: Jaejun KIM, Daejeon (KR); Ibrahim Bin Yasir, Daejeon (KR); Filippo Danilo Michelacci, Daejeon (KR); Kyuryang PARK, Daejeon (KR); Yongmin SEO, Daejeon (KR); Yeongryeol KIM, Daejeon (KR); Kyuyoung KIM, Daejeon (KR); Kyungmin HWANG, Daejeon (KR)

(21) Appl. No.: 19/202,730

(22) Filed: May 8, 2025

(30) Foreign Application Priority Data
Apr. 14, 2025 (KR) 10-2025-0047819

(51) Int. Cl. A61B 10/02; A61B 10/04; A61B 10/06

(52) U.S. Cl. CPC A61B 10/0275; A61B 10/04; A61B 10/06

(57) ABSTRACT

According to an embodiment of the present disclosure, a probe insertable into a body may include, a probe housing and a notch, wherein the notch includes a support portion and a notch body protruding from an upper surface of the probe housing, wherein the notch body includes at least one of a first groove, a second groove, and a third groove formed therein, and wherein the probe housing includes at least one of a first recess, a second recess and a third recess.

### CROSS-REFERENCE TO RELATED APPLICATIONS

This application claims priority under 35 U.S.C. § 119(a) to Korean Patent Application No. 10-2025-0047819, filed on Apr. 14, 2025 in the Korean Intellectual Property Office, the disclosures of which are incorporated by reference herein in their entireties.

### BACKGROUND

### 1. Field

The present disclosure relates to a medical instrument used for surgery within the body, and more particularly, to a probe that is insertable into the body through a trocar, is compatible with surgical instruments (such as graspers or forceps) within a body cavity, and is capable of performing real-time biopsy; and to a method of operating an image generation device including the probe.

### 2. Description of Related Art

Minimally invasive surgery, which is performed by inserting medical instruments into the body, has been widely adopted due to its various clinical advantages over open surgery, such as reduced pain, minimized bleeding, and shortened patient recovery time

To perform minimally invasive procedures, endoscopic, laparoscopic, and robotic surgical techniques are employed. These procedures involve making small incisions in the patient's body and inserting a trocar, which serves as a portal to allow various surgical instruments to access the internal cavity. Similarly, in cancer surgery, laparoscopic techniques offer a minimally invasive approach compared to traditional open surgery, thereby reducing postoperative complications and shortening the patient's recovery period.

In conventional minimally invasive surgeries, a separate biopsy instrument must be inserted to perform tissue sampling. Currently available laparoscopic probes are generally designed in the form of a rigid rod, which limits their maneuverability within the trocar. In other words, it is difficult to freely manipulate the probe once it has been inserted, and it is challenging to flexibly respond during tissue collection from specific regions. Furthermore, conventional laparoscopic probes do not have the capability to analyze tissue in real time. As a result, after performing a biopsy, the collected tissue must be sent to a pathology department for pathological interpretation in order to make a final diagnosis. This process takes a considerable amount of time and presents limitations in enabling medical personnel to make prompt clinical decisions.

### SUMMARY

An object of the present disclosure is to provide a real-time biopsy probe that is insertable into the body and operable within the body.

Another object of the present disclosure is to provide a biopsy probe that is compatible with conventional medical instruments, such as graspers or forceps, inside the body.

According to an embodiment of the present disclosure, a probe insertable into a body may comprise a probe housing and a notch. The notch may include a support portion and a notch body protruding from an upper surface of the probe housing. The notch body may include at least one of a first groove, a second groove, and a third groove defined therein, and the probe housing may include at least one of a first recess, a second recess and a third recess.

In addition, at least a portion of the notch body may be disposed between the first groove and the second groove, and the first and second grooves may be configured to receive a grasper in a second direction.

The first groove may be defined between a protrusion of the notch body and the support portion, and the first groove and the third groove may be configured to receive a grasper in a first direction.

The support portion may include at least one of a first support and a second support; a first inclined surface may be provided at one end of the first support, and a second inclined surface may be formed at an end of the second support. preferably, the first inclined surface is formed at a first end of the first support, and the second inclined surface is formed at a distal end of the second support, with reference to the optical window 41.

The second groove may be defined in a middle portion of the notch body, and the third groove may be disposed between a lower surface of the notch body and the probe housing.

When the grasper is received in the second direction, the grasper may come into contact with one surface of the first recess.

The second recess and the third recess may be arranged as a pair of grooves facing each other, and may be configured to allow a medical instrument to grip the probe housing from both sides.

The second inclined surface formed at the end of the support portion may be configured to facilitate withdrawal of the probe from a trocar.

The first inclined surface formed at one end of the support portion may be configured to facilitate insertion of the probe into a trocar.

When the grasper is held in the first direction, at least a portion of an outer surface of the grasper may come into contact with an upper surface of the probe housing and may be spaced apart from the first recess and the second recess.

The notch body may include an upper body in which the first and second grooves are formed and a lower body in which the third groove is formed.

A lower surface of the notch body, defined by the third groove, may come into contact with an inner surface of the grasper.

The third groove may extend across and connect the first groove and the second groove.

When the grasper is held in the second direction, at least a portion of an outer surface of the grasper may come into contact with a surface of either the first groove or the second groove.

At least a portion of an inner surface of the grasper may come into contact with an opposite surface of either the first groove or the second groove.

The inner surface of the grasper may refer to a surface of the grasper that comes into contact with the probe when the grasper grips or holds the probe.

The opposite surface of either the first groove or the second groove may refer to a surface of the probe that comes into contact with the grasper when the probe is gripped or held by the grasper.

A front end of the grasper may contact the first recess, and at least a portion of the grasper may pass through the protrusion of the notch body.

In an embodiment of the present invention, a probe insertable into a body includes a probe housing and a notch body disposed on the probe housing, wherein the notch body comprises a first groove, a second groove, and a third groove. The first groove and the third groove or the second groove and the third groove are configured to receive a grasper in different directions, and the third groove extends across a lower portion of the notch body to interconnect the first groove and the second groove. The third groove may further comprise a contact surface positioned at the lower portion of the notch body to receive a distal portion of the grasper. In addition, the probe housing may include a first recess, a second recess, and a third recess. The second and third recesses are disposed on opposite lateral surfaces of the probe housing to face each other, the first recess is formed to be compatible with a grasper, and the second and third recesses are respectively shaped and spaced apart to be compatible with forceps. Furthermore, inner surfaces of the second and third recesses may be positioned to come into contact with opposing surfaces of the forceps when the forceps grip the probe housing.

### BRIEF DESCRIPTION OF THE DRAWINGS

FIG. 1 is a diagram illustrating a microscope system according to an embodiment of the present disclosure.

FIG. 2 is a diagram illustrating an example of an environment in which the microscope system according to an embodiment of the present disclosure is used.

FIG. 3 is a perspective view illustrating a probe insertable into a body according to an embodiment of the present disclosure.

FIG. 4 is a front view of the probe according to an embodiment of the present disclosure.

FIG. 5 is a side view of the probe according to an embodiment of the present disclosure.

FIG. 6 is a diagram illustrating an example of operation of the probe according to an embodiment of the present disclosure.

FIG. 7 is another diagram illustrating an example of use of the probe according to an embodiment of the present disclosure.

FIG. 8 is a diagram illustrating a recess of the probe according to an embodiment of the present disclosure.

### DETAILED DESCRIPTION

The specific structural and functional descriptions of embodiments according to the concept of the present disclosure, as set forth in this specification, are provided merely for illustrative purposes to explain such embodiments. Embodiments in accordance with the concept of the present disclosure may be implemented in various forms and are not limited to the embodiments described herein.

Since embodiments in accordance with the concept of the present disclosure are capable of numerous modifications and variations in different forms, the embodiments are illustrated in the drawings and described in detail in this specification. However, it is not intended to limit the invention to the specific embodiments disclosed, and it is to be understood that all modifications, equivalents, and substitutions that fall within the spirit and scope of the invention are included.

The terms “first”, “second”, and the like may be used to describe various elements but should not be construed as limiting. Such terms are only used to distinguish one element from another. For example, without departing from the scope of the present disclosure, a first component may be referred to as a second component, and likewise, a second component may be referred to as a first component.

The terminology used herein is for the purpose of describing particular embodiments only and is not intended to be limiting of the invention. Singular forms are intended to include the plural unless the context clearly dictates otherwise. As used herein, the terms “comprise”, “have”, or any variations thereof, are intended to designate the presence of stated features, numbers, steps, operations, components, or combinations thereof, but do not preclude the presence or addition of one or more other features, numbers, steps, operations, components, or groups thereof.

Unless otherwise defined, all technical and scientific terms used herein have the same meanings as commonly understood by one of ordinary skill in the art to which this invention belongs. Terms that are generally defined in dictionaries should be interpreted as having a meaning that is consistent with their usage in the context of the relevant technical field, and not in an overly idealized or overly formal sense unless expressly so defined herein.

Further, the term “and/or” used herein is to be understood to include any and all possible combinations of one or more of the associated listed items. Moreover, the term “or” is intended to be inclusive, not exclusive, unless otherwise clearly indicated by the context. For example, the phrase “X uses A or B” is meant to include any of: X uses A; X uses B; or X uses both A and B.

The phrase “at least one selected from A and B” is to be interpreted broadly to include: (1) A; (2) at least one of A; (3) B; (4) at least one of B; (5) at least one of A and at least one of B; (6) at least one of A and B; (7) at least one of B and A; and (8) both A and B.

The singular forms used herein are intended to include the plural forms as well, unless the context clearly indicates otherwise. The terms such as “comprise” and “have” are to be interpreted as specifying the presence of stated features, steps, components, or combinations thereof, without precluding the possibility of the presence or addition of one or more other features, steps, components, or combinations thereof.

Prior to the detailed description of the drawings, it should be understood that the division of components described in this specification is merely for categorizing based on their primary functions. That is, two or more components described below may be integrated into a single component, or a single component may be further divided into multiple subcomponents based on functionality. Additionally, each component may perform part or all of the functions assigned to another component, or part of its functions may be performed entirely by a different component.

Moreover, in performing the method or method steps described herein, unless explicitly stated otherwise or required by context, the individual processes constituting the method are not necessarily limited to the order listed. The steps may be performed in the stated order, simultaneously, or in reverse order.

Hereinafter, preferred embodiments of the present disclosure will be described in detail with reference to the accompanying drawings.

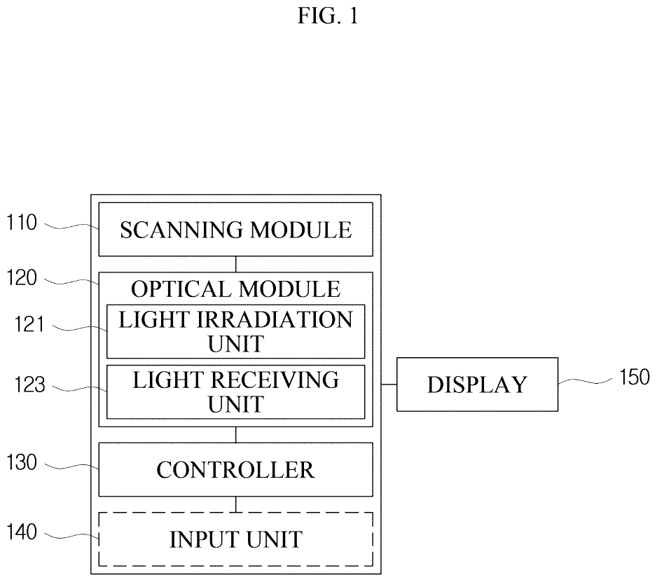

FIG. 1 is a diagram illustrating a microscope system according to an embodiment of the present disclosure.

Referring to FIG. 1, the microscope system may include a scanning module (110), a controller (130), and an optical module (120).

The scanning module (110) may irradiate light onto a target object either in contact therewith or at a predetermined distance from the object. Accordingly, the scanning module (110) may measure the interior of the object located within a preset distance from its surface.

For example, the preset distance may be varied by adjusting the focal length of a lens module, which will be described later, and may range from 0 μm to 250 μm.

In addition, the scanning module (110) may be a handheld device or a probe-type device that is insertable into the body. For example, the scanning module (110) may be an optical device in the form of an endoscope, a pen-type device, or a probe.

For example, a user may bring the optical device into direct contact with a target object or an area surrounding the target object, and the scanning module (110) may measure the interior of the target object located within a preset distance from the surface of the object.

In one embodiment, the scanning module (110) may be a confocal microscope(or endomicroscope) used in a clinical setting. For instance, a medical technician may bring the scanning module (110) into contact with a patient's skin surface, and the scanning module (110) may measure the condition of superficial cells located at a depth of approximately 50 μm from the contact surface.

Alternatively, for purposes such as cancer diagnosis or surgical margin assessment, a medical technician may bring the scanning module (110) into contact with an exposed region of a patient's body during an open or minimally invasive procedure. In such a case, the scanning module (110) may perform real-time measurements of internal biological tissue located at a depth of approximately 70 μm from the contact surface.

In order to effectively examine the pathological state of internal biological tissue, a fluorescent dye may be pre-injected into the body in the form of an injectable agent or the like. In this case, the scanning module (110) may irradiate light onto the target object, and an optical module (120), which will be described later, may detect a fluorescence signal returning from the target object.

Meanwhile, the scanning module (110) may perform a scanning operation in response to a drive signal supplied from a controller (130), which will be described later.

The controller (130) may be configured to control the overall scanning operation of the scanning module (110), such that the scanning module (110) performs scanning in accordance with a preset scanning pattern.

The controller (130) may apply a preset drive signal to the scanning module (110). The preset drive signal may include parameters such as frequency, voltage, and phase.

For example, the controller (130) may adjust the frequency, voltage, and phase in order to change the irradiation range of light performed by the scanning module (110).

Alternatively, the controller (130) may control the scanning module (110) to perform a scanning operation based on a drive signal input by a user.

The preset scanning pattern may vary, and the controller (130) may apply a drive signal corresponding to the preset scanning pattern to the scanning module (110).

In the present disclosure, the controller (130) may include at least one processor.

The optical module (120) is an optical system that irradiates light onto the scanning module (110) and detects signals returning through the scanning module (110).

In an embodiment of the present disclosure, the optical module (120) may be a confocal microscope(or endomicroscope) system, and the optical module (120) may be provided as a separate device, distinct from the scanning module (110).

The optical module (120) may include at least a light irradiation unit (121), (or light source unit) and a light receiving unit (123).

The light irradiation unit (121) may be a laser device that emits laser signals within a preset wavelength band. The laser device may be selected depending on the type of image to be observed from the target object, such as a reflective image, fluorescent image, or transmission image of the target object is to be observed.

For example, in an embodiment of the present disclosure, the laser device may emit at least one laser signal in the visible light region and/or the near-infrared region.

For example, in fluorescence imaging, laser signals having wavelengths such as 405 nm, 488 nm, 775 nm, and 785 nm may be used depending on the fluorescent dye employed. Here, the fluorescent dye may be used to distinguish pathological features of internal cells, blood vessels, or tissues, and may include dyes such as ICG, FNa, 5-ALA, and other dyes approved for medical use.

For instance, a tissue may be stained with a fluorescent dye responsive to a first wavelength band of laser signal and another fluorescent dye responsive to a second wavelength band, and the tissue may be irradiated with laser signals in the first and second wavelength bands through the laser device.

The fluorescent dye information may include information on at least one of PpIX (5-ALA), Hoechst, DAPI, Acridine orange (AO), Fluorescein Sodium (FNa), Fluorescein isothiocyanate (FITC), Propidiumiodide, acriflavine, Indocyanine Green (ICG), Pafolacianine, and Methylene Blue (MB).

The tissue image acquisition condition may include information on a laser used to acquire the tissue image (e.g., operation information on a laser output module of a microscope system). The laser information may include information on a first light having a first wavelength or a second light having a second wavelength. For example, the first wavelength may be included in one wavelength band of 400 to 410 nm, 480 to 500 nm, and 770 to 800 nm, and the second wavelength may be included in a wavelength band different from the wavelength band to which the first wavelength belongs, of 400 to 410 nm, 480 to 500 nm, and 770 to 800 nm.

In addition, the light irradiation unit (121) may apply an appropriate laser source to the scanning module (110) based on a signal input from an optical module controller (not shown).

Here, the optical module controller may control parameters such as the power of the laser signal emitted from the light irradiation unit (121) and the gain of the image.

For example, when the microscope system, according to an embodiment of the present disclosure, is used as a medical optical device, the power of the laser signal may be set to be equal to or less than 1 mW.

The optical module controller may be provided as part of the controller (130). In this case, the controller (130) may control the power of the laser signal emitted from the light irradiation unit (121), the image gain, and other parameters.

The light receiving unit (123) may be configured to detect signals returning from the target object in response to light irradiated from the light irradiation unit (121) through the scanning module (110).

Meanwhile, the light irradiation unit (121) and the light receiving unit (123) of the present disclosure may be integrated into a single unit within the optical module (120). That is, both the irradiation of light and the reception of signals reflected from the target object may be implemented through a single optical module (120).

Optionally, the microscope system may further include an input unit (140).

For example, the input unit (140) may serve as an input means for selecting an operation mode of the microscope system. In one example, the operation modes may include at least a first mode and a second mode that are preset. Here, the first mode may be a low-resolution mode or a search mode. Alternatively, the second mode may be a high-resolution mode or a zoom-in mode.

Accordingly, a user may select either the first mode or the second mode according to the intended purpose, and view an image of appropriate resolution through a display device (150).

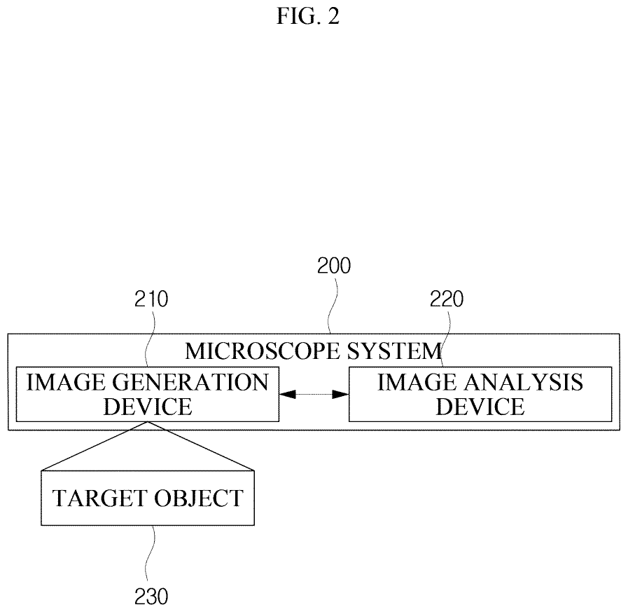

FIG. 2 is a diagram illustrating an example of an environment in which a microscope system according to an embodiment of the present disclosure is used.

Referring to FIG. 2, a microscope system (200) according to an embodiment of the present disclosure may include an image generation device (210) and an image analysis device (220). The image generation device (210) may include at least one of the scanning module (110), the optical module (120), the controller (130), the input unit (140), and the display device (150) as described in FIG. 1.

The microscope system (200) shown in FIG. 2 is merely exemplary, and each of the components may be implemented as a module within a single device, or may be physically separated and located at different places. In other words, the microscope system may refer not only to the microscope device itself, but also to the entire infrastructure used to inspect a target object through the operation of each of its components.

According to an embodiment of the present disclosure, the image generation device (210) may scan a target object (230) and generate images in real time.

In one embodiment, the image generation device may irradiate light onto the target object such that the path of the irradiated light follows a specific pattern. Through this, the path along which the light travels may represent a particular pattern.

The light irradiated onto the target object may follow a designated path for a certain period of time, and after the lapse of that time, overlapping may occur along the same path. That is, the certain period of time may correspond to the time required to complete the pattern.

For example, when an electrical signal input to the scanning module (110) includes a first drive signal that drives a component along the light path in one axial direction and a second drive signal that drives a component—such as the controller (130) or an element along the light path—in a direction different from the first axis, and when the frequencies of the first and second drive signals are different, the path of the light irradiated onto the target object may form a Lissajous pattern.

For example, the image generation device (210) may be a miniature fiber-optic scanner configured to observe the pathological state of biological tissue in real time in a laboratory or operating room.

In addition, the image analysis device (220) may be a device configured to perform real-time pathological diagnosis based on images generated by the image generation device (210).

For example, the image analysis device (220) may be an electronic device used by a medical professional for performing pathological diagnosis. Alternatively, the image analysis device (220) may be provided in the form of a module within such an electronic device. In another example, the image analysis device (220) may be a server configured to acquire images and perform analysis thereon.

The image generation device (210) and the image analysis device (220) may be connected via a network.

The network may include various wired or wireless networks, and the image generation device (210) and the image analysis device (220) may transmit and receive various types of information through the network.

For example, the image generation device (210) may transmit images generated by the image generation device (210) to the image analysis device (220) in real time via the network. The image generation device (210) and the image analysis device (220) may be located at different places.

For instance, the image generation device may be located in an operating room or laboratory where a surgical procedure or biopsy is being performed, while the image analysis device may be located in a pathology lab or examination room. In this case, the operating room and the pathology lab may be located in different hospitals or physical locations, enabling tele-pathology.

Meanwhile, when the image analysis device (220) is provided as a module within an electronic device assigned to a medical professional, or is provided in the form of a server, the image analysis device (220) may be implemented as a program or software application configured to perform pathological diagnosis based on images transmitted in real time from the image generation device (210).

Hereinafter, an image generation device (210) according to an embodiment of the present disclosure will be described.

The image generation device (210) according to an embodiment of the present disclosure may include a biopsy probe (300) that is insertable into the body.

The biopsy probe according to an embodiment of the present disclosure may include an optical biopsy probe or a digital biopsy probe.

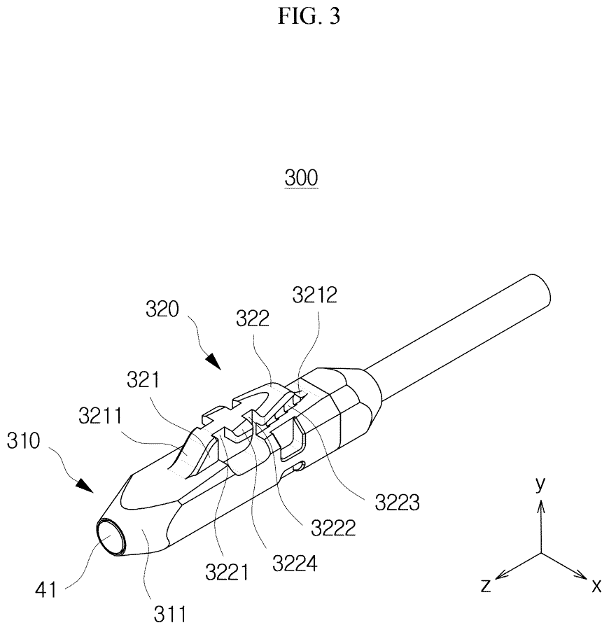

FIG. 3 is a perspective view illustrating a probe insertable into a body according to an embodiment of the present disclosure.

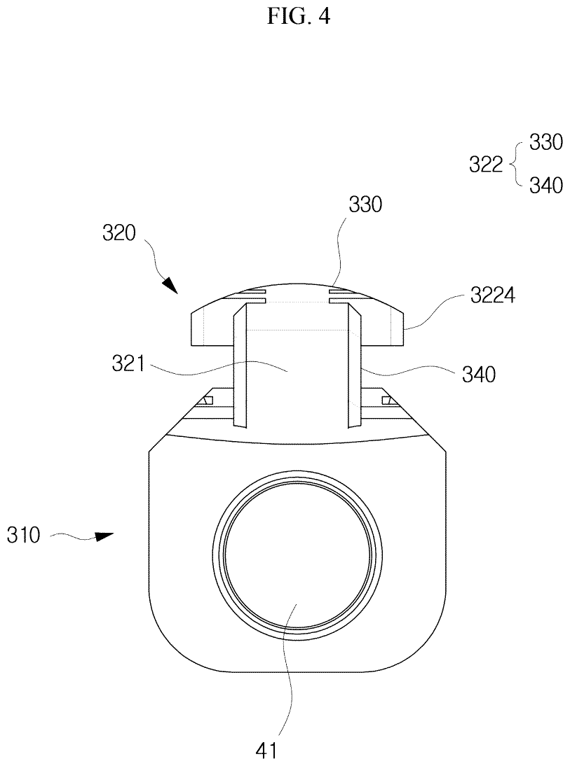

FIG. 4 is a front view of the probe according to an embodiment of the present disclosure.

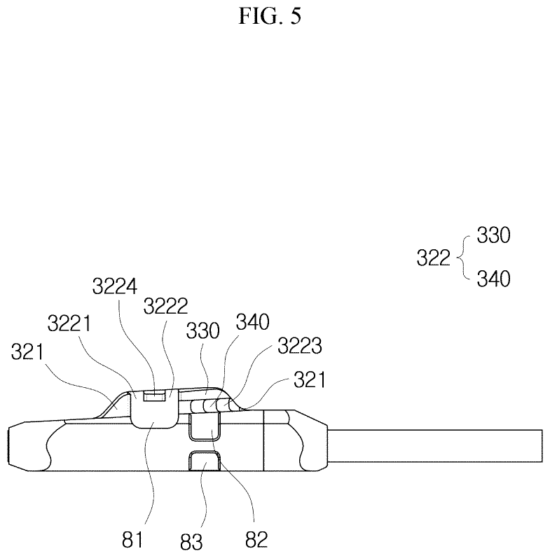

FIG. 5 is a side view of the probe according to an embodiment of the present disclosure.

Referring first to FIG. 4, a probe (300) according to an embodiment of the present disclosure may include a probe housing (310) and a notch (320).

The probe housing (310) defines the outer shape of the probe and may provide an internal space for accommodating components that perform various functions.

The probe housing may be made of various materials, such as plastic or stainless steel, to ensure stability and durability in the environments in which the probe is used. This allows the probe to efficiently interact with the target object into which it is inserted.

According to an embodiment of the present disclosure, a probe head (311) having an optical unit (41) disposed at one end of the probe housing may be included.

According to an embodiment of the present disclosure, the optical unit (41) may include an optical window composed of a glass substrate or an optical lens.

Referring to FIG. 3, the probe head (311) may include an opening to accommodate the optical unit (41).

The probe head (311) may be formed such that optical signals can be smoothly introduced through the opening, and an inclined surface may be formed to facilitate insertion into a trocar.

According to an embodiment of the present disclosure, the other end of the probe housing may include a cable or transmission path for connection with the image analysis device.

An inclined surface may be formed at the other end of the probe housing to facilitate withdrawal of the probe from a trocar. Through the inclined surface, the fiber tether and the probe may maintain airtightness with the external environment, thereby enhancing the bonding strength between the probe and the tether, while also serving to prevent the ingress of external contaminants (e.g., bodily fluids, air, or foreign substances).

Hereinafter, the probe housing (310) and the notch (320) will be described in greater detail.

According to an embodiment of the present disclosure, when the probe is inserted into the body, it may have a shape that is compatible with various medical instruments.

According to an embodiment of the present disclosure, the notch (320) is a structure formed on the probe housing, and is configured to facilitate the use of the probe with other surgical instruments (i.e., medical devices).

According to an embodiment of the present disclosure, the notch (320) may be formed to protrude from an upper surface of the probe housing and may include a shape in which the cross-sectional area changes discontinuously.

The notch may include a support portion (321) and a notch body (322) that protrude from the upper surface of the probe housing.

According to an embodiment of the present disclosure, the support portion may include a structure configured to support the notch on the upper surface of the probe housing

The support portion (321) may protrude from an upper surface of the probe housing.

According to an embodiment of the present disclosure, the notch (320) may include a notch body (322) that is connected to the support portion (321) and protrudes from the upper portion of the probe housing.

Referring to FIGS. 4 and 5, the notch body (322) may include an upper body (330) that forms an upper portion of the notch body, and a lower body (340) that forms a lower portion of the notch body.

According to an embodiment of the present disclosure, the support portion (321) may be at least partially connected to the notch body (322), and both the upper body (330) and the lower body (340) of the notch body (322) may also be connected to the support portion (321).

In this case, the support portion may include at least one support. For example, as shown in FIG. 3, when a pair of support portions is provided, both ends of the notch body (322) may be connected to the respective support portions of the pair.

Specifically, at least one support portion may be provided, and each of the at least one support portion may be connected to the notch body (322).

According to an embodiment of the present disclosure, a first support portion of the at least one support portion may be connected to one end of the notch body, and a first inclined surface (3211) may be formed on an upper surface of the first support portion.

In addition, among the at least one support portion, the second support portion may be connected to the other end of the notch body, and a second inclined surface (3212) may be formed on an upper surface of the second support portion.

The first inclined surface of the first support portion may have a shape in which a cross-sectional area increases from the probe head toward the notch body.

Similarly, the second inclined surface of the second support portion may have a shape in which a cross-sectional area increases from the distal end of the probe toward the notch body.

According to an embodiment of the present disclosure, the presence of at least one inclined surface on the support portion may allow the probe to more easily pass through a medical instrument such as a trocar when inserted into the body.

According to an embodiment of the present disclosure, at least one of the grooves (3221, 3222, 3223) may be positioned on the notch (320).

Specifically, the grooves may be formed in either the upper body (330) or the lower body (340) of the notch body (322). According to an embodiment of the present disclosure, the upper body (330) may extend from the upper surface of the support portion (321) in a direction different from the protrusion direction (y-axis) of the support portion.

Preferably, the different direction may be a direction perpendicular (x-axis) to the protrusion direction of the support portion. However, it should be understood that the invention is not limited thereto and that protrusion in various directions may also be included within the scope of the present disclosure.

As the notch body extends in a direction different from the protrusion direction of the support portion (hereinafter referred to as the “protrusion of the upper body”), it provides a body in which at least one groove (3221, 3222, 3223), as described later, may be formed, thereby allowing medical instruments such as a grasper or forceps to be effectively received therein.

At least a portion of the notch body may include a protrusion (3224) that extends in a direction different from the protrusion direction of the support portion.

As described above, the different direction may include various directions, including a direction perpendicular to the protrusion direction of the support portion.

Meanwhile, the lower body (340) of the notch body may be formed to protrude in the same direction as the protrusion direction of the support portion, and may connect the upper body (330) to the probe housing (310) while supporting the upper body (330).

In addition, a side surface of the upper body may be formed in a rounded shape that connects the upper surface and the lower surface.

Hereinafter, the first to third grooves will be described.

According to an embodiment of the present disclosure, the upper body of the notch body may include a first groove (3221) and a second groove (3222).

In addition, the lower body of the notch body may include a third groove (3223).

According to an embodiment of the present disclosure, the first groove (3221) may be formed between the protrusion (3224) of the upper body and the support portion (321).

In addition, the second groove (3222) may be formed between the protrusion (3224) of the notch body and the upper body.

Furthermore, at least a portion of the upper body may be present between the first groove (3221) and the second groove (3222).

Meanwhile, according to an embodiment of the present disclosure, the protrusion (3224) may be formed on a single side of the notch body with respect to its center.

Alternatively, the first groove (3221) and the second groove (3222) may be formed on both sides of the notch body (i.e., the upper body), such that the structure has a symmetrical shape with respect to a cross-section along the y-axis.

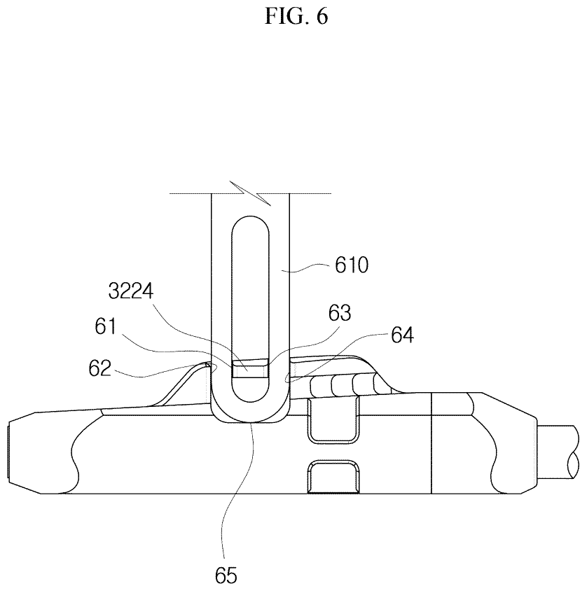

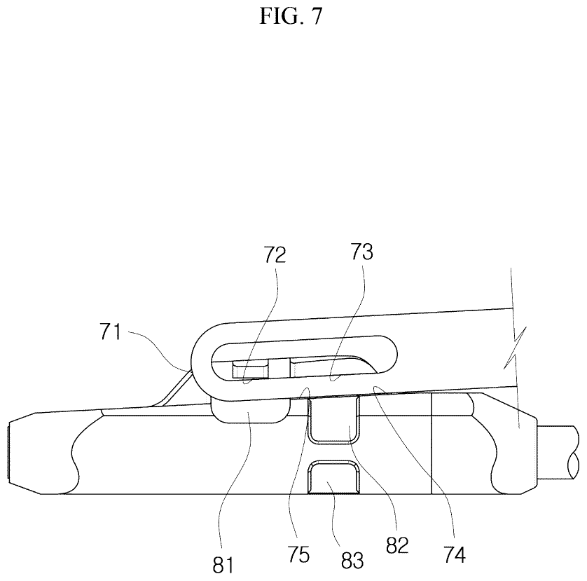

FIGS. 6 and 7 illustrate an embodiment showing the compatibility between a probe and a medical instrument according to an embodiment of the present disclosure.

Referring to FIG. 6, a grasper (610) used for intracavitary surgery may be received in a space defined by the first groove (3221) and the second groove (3222) formed in the notch body.

According to an embodiment of the present disclosure, the first groove may be configured to receive the grasper when the grasper is gripped in a vertical direction, which differs from the sensing direction of the probe (i.e., a second direction).

When the grasper is gripped in the second direction, at least a portion of an outer surface of the open-type grasper may come into contact with a surface of the first groove and/or the second groove.

In addition, at least a portion of an inner surface of the open-type grasper may come into contact with the opposite surface of the first groove (3221) and the second groove (3222).

A front end of the grasper may come into contact with the first recess, and at least a portion of the grasper may pass through a coupling portion (coupling slot).

Further, the inner surface of the grasper may come into contact with at least one surface of the outer surface of the protrusion (3224) of the notch body. A lower surface of the protrusion (3224) may be spaced apart from the inner surface of the grasper by a predetermined distance.

Additionally, the first groove (3221) and the second groove (3222) may be recessed in a specific direction such that the bottom surfaces of the two grooves are shared, thereby forming a U-shaped groove. Specifically, since the bottom surfaces of the first and second grooves continue from the same location, a curved or U-shaped groove may be formed.

Moreover, a distal end of the grasper may come into contact with at least a portion of the bottom surface (65) defined by the first groove (3221) and the second groove (3222).

Through this configuration, the first groove (3221) and the second groove (3222) may function as a single, continuous structure with a connected bottom surface, thereby providing a stable contact surface when coupled with a specific object, such as a grasper.

In addition, the U-shaped groove may assist in position alignment during insertion or coupling and may perform a guiding function that induces movement in a specific direction while maintaining mechanical strength.

When the grasper (610) is received in the first groove (3221) and the second groove (3222), the lateral rotation and forward/backward movement of the probe may be restricted, thereby enhancing the control stability for the medical operator.

According to an embodiment of the present disclosure, the notch body (322) may further include a third groove (3223). As described above, the third groove (3223) may be formed in the lower body.

The third groove (3223) formed in the lower body may be connected to the second support portion of the at least one support portion.

Through this configuration, a portion of the grasper may come into contact with the second support portion and the lower body via the third groove.

Referring to FIG. 5, according to an embodiment of the present disclosure, the third groove (3223) may be formed between the lower body of the notch body and the probe housing.

When the grasper (610) is received in the third groove and the first groove (3221), the grasper may be gripped in a direction aligned with the scanning direction of the probe.

In this case, the third groove may be connected across the first groove and the second groove.

The third groove may be formed in a direction different from that of the first and second grooves, and may extend in the longitudinal direction of the probe.

For example, the first groove (3221) and the second groove (3222) may be recessed in a vertical direction, while the third groove (3223) may be formed in a different direction to define an independent path within the lower body of the notch body.

The third groove (3223) may have one end connected to a support portion located at the opposite end of the notch body, and the other end of the third groove may be connected to a region in which the second groove (3222) is formed. That is, the third groove may be formed to extend from the support portion at the distal end of the notch body toward the probe head.

Due to this structural difference, the third groove may be configured to receive the grasper when the grasper is gripped in the longitudinal direction of the probe (i.e., the target sensing direction, or first direction).

Referring to FIG. 7, when the grasper is gripped horizontally according to an embodiment of the present disclosure, at least a portion of an outer surface of the grasper (i.e., a surface located on the outside of the grasper jaws, opposite to the inner surface) may come into contact with upper surfaces (74, 75) of the probe housing, and may be spaced apart from the first recess (81) and the second recess (82).

In addition, a lower surface (73) of the notch body formed by the third groove, and a lower surface (72) of the protrusion, may come into contact with an inner circumferential surface of the grasper.

Furthermore, as the third groove is connected across the first groove and the second groove, the grasper may be gripped in parallel with the target scanning direction of the probe.

In this case, the contact surface of the grasper may have a toothed structure to more securely grip the probe.

Correspondingly, a surface of the third groove may include a step structure that engages with the toothed structure of the grasper, thereby increasing frictional force.

Meanwhile, according to an embodiment of the present disclosure, a distal end surface of the grasper may come into contact with a side surface (71) of the support portion. The side surface (71) of the support portion may have a rounded shape similar to the shape of the distal end of the grasper.

In addition, the third groove (3223) may be less recessed than the first groove (3221) and the second groove (3222), such that when the grasper is received vertically in the first and second grooves, the third groove contributes to grip stability of the grasper.

Hereinafter, the first to third recesses will be described.

### Probe Housing

According to an embodiment of the present disclosure, the probe housing (310) may include a first recess (81).

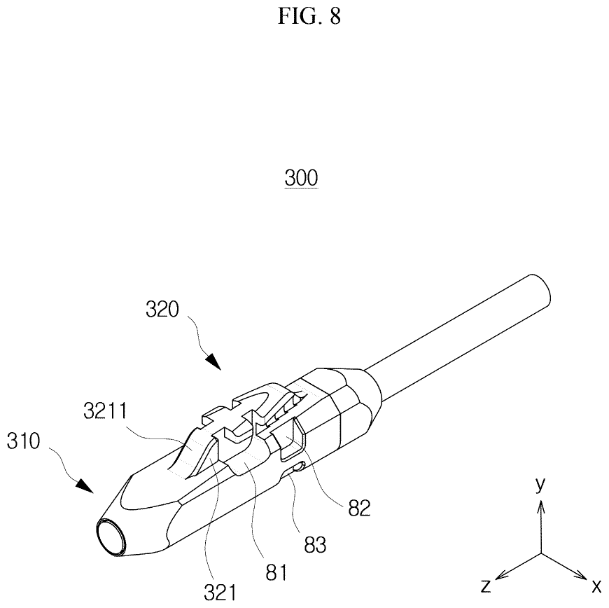

FIG. 8 is a diagram illustrating a recess of the probe according to an embodiment of the present disclosure.

According to an embodiment of the present disclosure, the first recess (81) may have a depressed shape formed inwardly from the upper side of the probe housing in a direction parallel to the height (y-axis direction) of the probe.

At least a portion of a surface of the first recess (81) may be connected to the first groove (3221) and/or the second groove (3222).

Accordingly, a virtual line extending in the height direction of the probe from at least a portion of the surface of the first recess (81) may pass through at least one of the first groove or the second groove.

A surface of the first recess (81) may be designed to come into contact with an end surface of the grasper. Among the surfaces formed by the first recess (i.e., a first surface, a second surface, and a third surface), at least one of the surfaces may come into contact with a corresponding end surface of the grasper.

Specifically, the first surface of the first recess (81), which corresponds to the bottom surface of the recess, may include a fixing means configured to control the movement of the probe.

In addition, the second surface and third surface of the first recess (81), which correspond to the sidewalls of the recess, may prevent lateral rotation and forward/backward movement of the probe.

According to an embodiment of the present disclosure, the probe housing (310) may further include a second recess (82) and a third recess (83).

According to an embodiment of the present disclosure, the second recess (82) and the third recess (83) are depressions designed to allow a medical instrument, such as forceps, to grip the probe housing and facilitate adjustment at various angles.

Specifically, the second recess (82) and the third recess (83) may be arranged such that two grooves face each other, allowing the medical instrument to securely grip the probe from both sides in a balanced manner.

For example, one leg of the forceps may be received in the second recess (82), and the other leg may be received in the third recess (83), thereby enabling a firm grip on the probe.

This structure allows the forceps to securely hold the probe while also enabling fine adjustment when necessary.

In addition, since the two recesses are symmetrically formed, the forceps can apply a balanced grip force when holding the probe, thereby allowing smooth movement and rotation of the probe during surgery.

According to an embodiment of the present disclosure, a biopsy probe may be provided that is compatible with conventional medical instruments and is capable of analyzing tissue in real time.

In addition, by utilizing a microscope system including the biopsy probe, tissue examination can be performed without inserting a separate biopsy instrument, thereby enabling real-time diagnosis and supporting medical personnel in making prompt decisions during surgery.

Furthermore, since the shape of the biopsy probe according to an embodiment of the present disclosure is designed to be easily compatible with existing medical instruments, the biopsy probe can be effectively manipulated within a body cavity.

Meanwhile, in an embodiment of the present invention, the aforementioned components—such as the first to third grooves and the first to third recesses—may be symmetrically formed with respect to a cross-section of the probe.

### DESCRIPTION OF REFERENCE NUMBER

300: probe
310: probe housing
311: probe head
320: notch
321: support portion
322: notch body
3221: first groove
3222: second groove
3223: third groove
3224: protrusion
41: optical unit
81: first recess
82: second recess
83: third recess
3211: first inclined surface
3212: second inclined surface

What is claimed is:

1. A probe insertable into a body,
the probe comprising:
a probe housing; and
a notch,
wherein the notch includes a support portion and a notch body protruding from an upper surface of the probe housing,
wherein the notch body includes at least one of a first groove, a second groove, and a third groove formed therein, and
wherein the probe housing includes at least one of a first recess, a second recess and a third recess.

2. The probe of claim 1,
wherein at least a portion of the notch body is disposed between the first groove and the second groove, and
wherein the first groove and the second groove are configured to receive a grasper in a second direction.

3. The probe of claim 1,
wherein the first groove is formed between a protrusion of the notch body and the support portion, and
wherein the first groove and the third groove are configured to receive a grasper in a first direction.

4. The probe of claim 1,
wherein the support portion includes at least one of a first support and a second support,
wherein a first inclined surface is formed at one end of the first support, and
wherein a second inclined surface is formed at an end of the second support.

5. The probe of claim 1,
wherein the notch body includes an upper body in which the first groove and the second groove are formed, and a lower body in which the third groove is formed.
wherein the second groove is formed in a middle portion of the upper body, and
wherein the third groove is formed between a lower surface of the upper body and the probe housing.

6. The probe of claim 1,
wherein, when the grasper is received along the second direction, the grasper contacts an inner surface of the first recess.

7. The probe of claim 1,
wherein the second recess and the third recess are disposed to face each other as a pair of grooves, such that a medical instrument can grip the probe housing from both sides.

8. The probe of claim 1,
wherein the second inclined surface formed at the end of the support portion is configured to facilitate withdrawal of the probe from a trocar.

9. The probe of claim 1,
wherein the first inclined surface formed at one end of the support portion is configured to facilitate insertion of the probe into a trocar.

10. The probe of claim 1,
wherein, when the grasper is held in the first direction, at least a portion of an outer surface of the grasper comes into contact with an upper surface of the probe housing,
and is spaced apart from the first recess and the second recess.

11. The probe of claim 1,
wherein the notch body includes an upper body in which the first groove and the second groove are formed, and a lower body in which the third groove is formed.

12. The probe of claim 11,
wherein a lower surface of the notch body defined by the third groove comes into contact with an inner surface of the grasper.

13. The probe of claim 11,
wherein the third groove extends across and connects the first groove and the second groove.

14. The probe of claim 1,
wherein, when the grasper is held in the second direction, at least a portion of an outer surface of the grasper comes into contact with a surface of either the first groove or the second groove.

15. The probe of claim 1,
wherein at least a portion of an inner surface of the grasper comes into contact with an opposite surface of either the first groove or the second groove.

16. The probe of claim 1,
wherein a front end of the grasper contacts the first recess, and at least a portion of the grasper passes through the protrusion of the notch body

17. A probe insertable into a body, comprising:
a probe housing; and
a notch body disposed on the probe housing,
wherein the notch body comprises a first groove, a second groove, and a third groove,
wherein the first groove and the third groove or the second groove and the third groove are configured to receive a grasper in different directions, and
wherein the third groove extends across a lower portion of the notch body and interconnects the first groove and the second groove.

18. The probe of claim 17,
wherein the third groove comprises a contact surface disposed at a lower portion of the notch body and positioned to receive a distal portion of a grasper.

19. A probe insertable into a body, comprising:
a probe housing including at least a first recess, a second recess, and a third recess,
wherein the second recess and the third recess are disposed to face each other on opposite lateral surfaces of the probe housing, and
wherein the first recess is formed at the probe housing and is dimensioned to be compatible with a grasper,
and the second recess and the third recess are respectively shaped and spaced apart to be compatible with forceps.

20. The probe of claim 19,
wherein inner surfaces of the second recess and the third recess are respectively positioned to come into contact with opposing surfaces of forceps when the forceps grip the probe housing.
